(19) United States
(12) Patent Application Publication
(10) Pub. No.: US 2004/0228800 A1
(43) Pub. Date: Nov. 18, 2004

(54) Non-invasive identification of patients at increased risk for myocardial infarction

(75) Inventor: Eckhard Alt, Ottobrunn (DE)

Correspondence Address: DONALD R. GREENE, P.O. BOX 12995, SCOTTSDALE, AZ, 85267, US

(73) Assignee: SciCoTec GmbH, Munich (DE)

(21) Appl. No.: 10/439,272

(22) Filed: May 15, 2003

(51) Int. Cl. A61K 49/00

(52) U.S. Cl. 424/9.52

(57) ABSTRACT

A method of identifying a patient at increased risk for myocardial infarction utilizes detection of a site or sites of pathologic vascular function in the coronary circulation of the patient. In the method, microbubbles are formed, each consisting of an encapsulated biocompatible gas characterized by rapid dissolution in blood, and the microbubbles are labeled with a prescribed antibody. These microbubbles are then injected into the patient's circulation to allow them to preferentially attach to a site of antigens specific to the pathologic vascular function in the coronary circulation. Ultrasonic energy is applied to the patient's coronary circulation at a level sufficient to burst the microbubbles that are preferentially attached to the targeted site and allow the encapsulated gas to escape from the burst microbubbles for absorption by the blood. Location of the targeted site is identified by detecting a signal representing reflectance of ultrasonic energy from the site.

### BACKGROUND OF THE INVENTION

The present invention relates generally to medical procedures, and more specifically to methods for identifying patients who, because of unstable plaque on blood vessel walls, are at increased risk for myocardial infarction or ischemia resulting in pain or dysfunction in other body organs or parts, and for achieving the identification by non-invasive techniques.

Despite considerable progress in methods of treatment of patients with acute myocardial infarction (MI), a mortality rate between 20-30% remains at this time because most victims die from electrical fibrillation before reaching the hospital. In contrast, the mortality rate reported in clinical studies for patients reaching the hospital with an acute MI ranges between 3 and 7%, this being relatively low since the availability of intensive care treatment, defibrillation, and revascularization procedures such as thrombolytic therapy or interventional cardiology to open an occluded artery exists in a clinical setting. Thus, it is quite apparent that if the occurrence of a myocardial infarction could be prevented, especially outside the clinical setting, it would result in a saving of many lives notwithstanding the many advances that have been made in hospital emergency room treatment methods for MI patients.

More than 50% of acute myocardial infarctions occur in patients who have had no or only limited and unspecific previous symptoms. The explanation is that a narrowing of a coronary artery with plaque build-up to a degree of less than 50% stenosis causes no symptoms, since the circulatory reserve in the coronaries is sufficiently large that the blood flow is adequate even through such a restricted artery. For many of these patients, the first recognition of coronary heart disease is a myocardial infarction.

The pathologic physiology of the disease process is a slow but consistent build-up of arteriosclerotic plaque over a prolonged period of time. Despite an absence of symptoms, the plaque may suddenly rupture and expose sub-endothelial substrate to the flow of blood. This event activates a coagulation cascade, with formation of platelets and fibrin deposits on the exposed substrate. In addition, inflammatory processes induced by leukocytes and monocytes promote plaque instability, and metalloproteinases degrade the matrix and expose the sub-endothelial layers to the blood stream. Thus, the protective layer of endothelial cells normally found in a healthy artery is compromised or interrupted, and an increasing adhesion of cells and thrombus formation takes place.

Currently, a major aim in the field of cardiology is to provide a capability to identify and distinguish patients with a serious risk of unstable plaque that can cause a sudden myocardial infarction, from patients whose 50% narrowing of a coronary artery is benign and relatively risk-free over the long term. A lesion that narrows the arterial lumen by 50% is normally not a case for an interventional cardiology procedure such as balloon angioplasty or stent implantation. But the presence of unstable plaque and accompanying risk of myocardial infarction does call for an intervention such as stenting. The current availability of immuno-supressant and anti-coagulation drug-coated stents make such a procedure particularly beneficial.

Among the more recent techniques developed to detect unstable plaque is that of performing temperature measurements at the site of the plaque. It is known that the inflammatory process ongoing inside of an arteriosclerotic plaque produces a local temperature increase ranging between 0.2° and 2.0° C., depending on the extent of the ongoing inflammation. Small probes with thermocouple link elements are used for local detection of the temperature disparity, in the attempt to distinguish a vessel undergoing inflammation from a normal arterial vessel wall. But the value of using temperature measurements to identify possibly unstable plaque and distinguish it from a harmless build-up with no tendency to rupture, carries with it a range of risks, including possibly lethal complications, associated with a need for coronary arterial access to introduce a catheter and inject contrast dye. The patient faces a difficult decision whether to undergo a relatively risky interventional procedure where no apparent symptoms but some risk factors are present. In addition, plaque inflammation and increased temperatures can be found also in plaques that are not prone to become vulnerable and unstable with the risk of thrombus build-up and subsequent infarction.

Current non-invasive techniques for detecting unstable plaque include patient testing to indicate portions of the coronary circulation where stenosis may exceed 50% and where an ischemic response is occurring with physical exercise. Indeed, virtually all current non-invasive procedures seeking to identify patients at-risk for unstable plaque aim toward the demonstration of ischemia. Exercise stress test including EKG changes and subjective symptoms, echo cardiography with stress and demonstration of an area of less contraction under ischemia, or nuclear test including sestamibi Technetium 99 or thallium to show a fixed perfusion defect, use such means.

A recent technique for demonstrating the presence of coronary ischemia involves injecting tiny bubbles—microbubbles—composed of nitrogen encapsulated in a polylactic acid (PLA) and protein shell into the venous circulation, as described, for example, in U.S. Pat. Nos. 6,193,951 and 6,482,518. These microbubbles are approximately 3 or 4 micrometers (μm, or microns) in diameter, sufficiently small—even smaller than red blood cells—to pass through the capillary bed of the lungs. Thus, if such nitrogen-filled contrast bubbles or other small aerial enhanced contrast media are injected into the venous circulation, they will pass through the lungs and be present in the pulmonary and systemic circulation.

By application of ultrasound with power Doppler, first and second harmonic imaging, the tissue perfusion of the myocardium can readily be demonstrated. The bubbles preferentially stay in the arterial and capillary circulation and their flow through an artery with significant obstruction is limited—such with physical exercise—and their reduced presence distal of a stenosis is indicative of ischemia in the respective coronary circulation. They and their location in the myocardial tissue that is perfused by these bubbles is detected by application of ultrasonic energy which tends to destroy their shells and yields a reflectance level—a contrast ratio—indicative of the presence in the tissue. This myocardial contrast tissue cardiography has been revealed with sufficient success that myocardial opacification from a venous injection is proceeding toward clinical routine application. The principle is that the microbubbles of such small diameter scatter these waves. The backscatter information obtained from application of ultrasound varies greatly, however, because of differences in bubble size, stability and concentration. But it is known that power Doppler and harmonic imaging can produce a relatively stable signal and serve to enhance the signal detection.

The general principle of myocardial contrast echocardiography is presented in Progress in Cardiovascular Diseases, vol. 44. No. 1, July-Aug. 2001, pp.1-11. Aside from an increasing importance of myocardial opacification, from venous injection, and the consequent application of myocardial contrast echocardiography, it has been desired to identify certain target sites within the body by means of ultrasound contrast agents. The principle involves detecting inherent chemical or electrostatic properties of the microbubble shell during circulation that result in their preferential retention at the sites of interest with specific disease processes. For example, leukocytes have a tendency to absorb bubbles by phagocytosis and digest them. If leukocytes are activated and bound to an inflammatory site, for example, finding of an increased presence of leukocytes at a certain site from detection of an increased reflection pattern attributable to the phagocytosed bubbles in the leukocytes is indicative of inflammation.

Another strategy is to detect the attachment of specific antibodies or other ligands linked to the microbubble surface that react with certain antigenic structures. Despite many attempts in the past to detect microbubbles bound to a specific antigenic site, none of these attempts has, thus far, been successful, apparently because the site of inflammation or unstable plaque presents a limited exposed surface, typically only several square millimeters.

A pre-condition for reliable detection of a signal in the myocardial tissue is to apply an appropriate predetermined level of mechanical energy to destroy the majority of all injected bubbles. Normally, this mechanical energy is in the range of from 0.5 to 0.7 mechanical indices. Destruction of the shell of the bubble and absorption of the gas contained therein in the blood produces an instant change in echo contrast. The majority of contrast bubble agents currently used contain peroxyfluorocarbons because of its ease of entrapment in the shell and its reduced solubility in blood.

One of the earliest methods of this type is described in U.S. Pat. No. 5,334,381, which discloses the use of liposomes as contrast agents for ultrasonic energy, and methods for their preparation. These liposomes are made from phosphorylcholin and the encapsulation of various gases is suggested. However, the leakage of gas from the shell is such that only peroxyflurocarbon gases are considered to be suitable for encapsulation in the shell since they are less prone to leak. On the other hand, low leakage is associated with low solubility, which has the disadvantage that the low solubility gas released upon disruption of the bubble remains in the blood and makes the change in acoustical impedance inadequate for reliable detection.

Other attempts made in the past have included studying microbubbles targeted to attach to integrins expressed on the surface of altered microvascular perfusion. These signals with liposomes in peroxyflurocarbon gas are not strong enough to be detected in humans in a myocardial circulation—only animal preparations using a microscope have been conducted thus far because of the limitations of the signaling bubble intensity. See, for example, “Non-invasive assessment of angiogenesis by ultrasound in microbubbles targeted . . . ” by Howard Leon Poy in Circulation 2003, no. 107, pp. 455-460.

An article titled “Non-invasive ultrasound imaging of inflammation using microbubbles targeted to activated leucocytes” by Lindner in Circulation 2000, no. 102, pp. 2745-2750, describes enhancing interaction by complement activation. The risk of such complement activation, however, is an unpredictable disseminated intravascular coagulation.

In vivo targeting of acoustically reflective liposomes for intravascular and transvascular ultrasonic enhancement has studied the acoustics with small liposomes of less than one micron where the liposomes can target to fibrin-coated surfaces. While the ultrasound detection of the labeled liposomes with antibodies against antifibrinogen targeted to thrombi and against intracellular adhesion molecules (ICAM-1) is feasible in the animal model and is suitable to detect early stages of arteriosclerotic processes, the reflection from such liposomes not filled with gas is difficult to obtain in patients in a transthoracic manner. In this regard, the reader is referred to an article entitled “In vitro targeting of acoustically reflecting liposomes for intravascular transfer and for ultrasonic enhancement,” published in the Journal of the American College of Cardiology (JACC) (1999), no. 33, pp. 867-875.

Other attempts to detect tiny amounts of bubbles in these circumstances have involved study of the negative charge of certain bubbles retained within capillaries via complement-mediated attachment to the endothelium. See, for example, “Influence of microbubble surface charge on capillary transit and myocardial contrast enhancement” by Nicholas Fisher in JACC (2002), no. 40, pp. 811-819. This article indicates that the adhesion of charged microbubbles to altered endothelial structures can be obtained in a small animal model. But in patients whose thoracic cage and heart are of normal human adult dimensions, the bubble signal intensity in such a setting is inadequate for reliable detection.

### SUMMARY OF THE INVENTION

A principal aim of the present invention is to provide a novel method to identify patients at risk with unstable plaque by a non-invasive technique, and to select those of the identified patients whose risk of MI is sufficiently great to justify the subsequent cost and risk associated with an interventional diagnostic procedure including coronary angiography, temperature measurements, optical coherent identification, and endovascular ultrasound, by way of example.

Another aim is to identify by improved non-invasive techniques, patients in whom ischemic heart disease with obstruction of the coronaries is or is likely to be present together with unstable plaque, and a consequent increased risk of myocardial infarction.

The invention takes advantage of the concept of using tiny nitrogen or other appropriate gas-filled bubbles that can circulate in the patient's arterial and venous system within a double shell, that is, a shell having two layers—namely, an inner layer of a polymer such as polylactic acid (PLA) and an outer layer of protein such as albumin. This is described, in and of itself, in patent U.S. Pat. No. 6,193,951. The contrast-producing nitrogen bubbles can be detected by echocardiographic techniques. Perfusion with these bubbles, coupled with sonication, causes the bubbles to rupture and emit a signal that can be detected by second harmonic imaging. Currently, this method is used to detect obstruction of blood flow and ischemia not only in the myocardium tissue as a whole, but also to differentiate sub-endocardial, epicardial and total myocardial ischemia. However, to identify unstable plaque, it is necessary to bind directly to sites where an unstable vessel wall situation exists. The location is identified by the preferential adhesion of the bubble to a site of protein expressed by an unstable plaque, as the bubbles are carried through the patient's circulatory system.

To that end, and according to an aspect of the present invention, the microbubble shell is loaded with antibodies selected to react with the expressed antigen protein at the target sites as the bubbles are carried through the blood circulation system, so as to bind to those sites. The shell of each bound bubble is then disrupted by subjecting the region to ultrasonic energy, to release the encapsulated gas and produce a contrast change in the detected signal reflected from the sites for identification of sites of unstable plaque.

But more is needed to assure reliable site detection. The present invention relies on formation of bubbles with a double-layer shell that encapsulates a highly soluble gas adapted to undergo rapid dissolution in the blood; loading the bubble's shell with antibodies selected to enhance binding of the bubbles, after injection into the patient's circulation, at sites that express the antigen protein indicative of inflammation; destruction of bubbles remaining in the circulatory system after a predetermined period of circulation that will allow relatively large quantities of the bubbles to bind to the target sites; to avoid a masking effect from still circulating bubbles on reliable identification of the unstable plaque sites in the signal detection phase; performing this destruction by sonication along vessels that carry large amounts of blood; repeating bolus injections of, or continuously injecting, bubbles to overcome retention of bubbles in the liver, pancreas and spleen; and, after applying ultrasonic waves to the regions of the circulatory system of primary suspicion as harboring unstable plaque to measure signal reflectance therefrom, detecting the difference in reflectance, rather than the absolute reflectance, to optimize the ratio of the bound bubble-emitted signal-to-background noise, and thereby, the contrast ratio that identifies the sites.

The steps necessary to enhance the detection of the target sites include detecting decay of the signal over a specified short period of time, rather than detecting the absolute level of reflected signal. For example, if a train or burst of, say, 4 to 10 ultrasonic pulses is applied within a short period of, say, 10 to 300 ms, the first or second pulse will usually destroy the bubble. The change in signal reflection over the entire train of pulses is then detected to more reliably identify presence of unstable plaque. This may be likened to the operation of a differential amplifier, which serves to eliminate the presence of background noise to enhance the level and purity of the output signal. Relatively high solubility of the bubble-encapsulated gas, and rupture of the bubble to release the gas at a defined level of mechanical energy applied to the bubble, are prerequisites for detection of decay of the signal over time. Applicant's investigation has ascertained that these pre-conditions can be fulfilled with the type of bubbles described in the aforementioned U.S. Pat. No. 6,193,951.

Certain specific considerations must be observed for the method of unstable plaque detection to be deemed clinically reliable. Among these considerations are that the size of the bubble must properly match the mechanical forces of the site attraction for binding, and the forces exerted in the binding must exceed the mechanical forces generated by the blood flow to resist a tendency to dislodge bubbles from the site. Thus, a bubble size less than 3 microns, and optimally equal to or less than one micron, is beneficial because it reduces the mechanical forces acting upon such a bound bubble of such small size. Even with bubbles of such small size, the bubble shell surface can expose a sufficient number of ligands to realize an effective and reliable binding of the bubble to the specific antigen expressed at an unstable plaque site. If the number of antigens bound to the surface of the bubble is too large, a self-perpetuation of micro-emboli occurs; especially if the FAB fragment that activates complement is present in the bindings. Accordingly, only FAC fragments are sufficient to constitute the antibody structure on the surface of the bubble.

Beyond considerations of the binding and the mechanical energy involved, an aspect of the invention of major importance lies in incorporating the correct antibodies into the bubble's shell. When a plaque becomes unstable, expressed by a pathologic vascular function, the endothelial lining at its site is no longer present, or is damaged or incomplete. This triggers monocytes that adhere to the incomplete endothelium, with an increased binding of cells to the characteristic CD11B. In a subsequent phase, platelets are activated and deposited on the plaque in addition to the monocytes. These activated platelets are typically characterized by a surface marker known as CD62 or CD63. Also, fibrinogen bridges bind between the activated platelets, and can be detected by anti-fibrinogen antibodies.

Thus, according to the invention, a method of identifying a patient at increased risk for myocardial infarction relies on detection of pathologic vascular function and of unstable plaque in the coronary circulation of the patient. Microbubbles comprising biocompatible, highly soluble gas encapsulated in a double-layer shell are formed and labeled with antibodies of an inflammatory-specific antigen. The microbubbles are injected into the patient's venous circulation to enter the pulmonary circulation and thereafter, the coronary circulation where, by virtue of the antibody labeling, they undergo preferential binding to sites of the antigen expressed by exposed sub-endothelial structures, and thus, unstable plaque. Ultrasonic energy is applied to the patient's coronary circulation at a level sufficient to burst the microbubbles bound to the target site, enabling escape of the encapsulated gas for absorption by the blood. The signal level decay representing relative change in reflectance of ultrasonic energy from the site over a preset interval of time is differentially detected as indicative of the presence and location of a site of unstable plaque in the locale from which the signal emanates.

The bubbles may be characterized by (a) propensity to undergo binding specific to sites of unstable plaque in the systemic circulation, (b) force of the binding to a site of unstable plaque exceeding mechanical forces exerted on the bubble by blood flow, (c) the defined level of mechanical energy required to burst the bubble, and (d) rapid solubility of the gas released by the burst bubble.

In a method of identifying a site of pathologic vascular function in a patient's blood vessel, the bubbles are injected and undergo preferential binding, as above, at each such site. The bound bubbles are then subjected to a train of ultrasound pulses sufficient to produce the defined level of mechanical energy for rupture. The bubbles are further characterized by a detectable decaying signal upon rupture and dissolution of the gas in the blood, to optimize the ratio of bound bubble-emitted signal-to-background noise. The bubbles are allowed to undergo systemic circulation for a period of time sufficient for preferential binding of bubbles to sites of pathologic vascular function in the arteries. Bubbles that are still circulating after the predetermined period of time are selectively destroyed by sonication at a site different from the target site to increase the sensitivity for detecting the bound bubbles in the vessel.

The pathologic vascular function may be a ruptured plaque, or the site may be one with a pathologic endothelial function. A specific method to detect a site of pathologic vascular function in a patient's arterial circulation system may include intravascular application of gas filled echo contrast bubbles having a surface conjugated with a specific antibody against CD11b at the site, and identifying the location of the site from the bubbles bound thereto by application of ultrasound. Or the antibody used may be specific against CD 62 or CD 63. Or the antibody may be against more than a single antigen present at a site of pathologic vascular function. It will be understood these are merely illustrative examples.

### BRIEF DESCRIPTION OF THE DRAWING

The above and still further aims, objectives, features, aspects and attendant advantages of the present invention will become apparent to those skilled in the art from a consideration of the following detailed description of a best mode presently contemplated for practicing the invention, by reference to certain preferred methods of the invention, taken in conjunction with the accompanying figures of drawing, in which:

FIG. 1 is a cross-section of a microbubble formed according to the invention;

FIG. 2 is a perspective transparent view of a coronary artery showing a site of unstable plaque to which the injected microbubbles are bound;

FIG. 3 is a graph, shown in conjunction with a train of ultrasonic pulses, illustrating the decay of reflectance as a differential detection (delta) attributable to the rapid dissipation of reflectance; and

FIG. 4 is a graph illustrating the energy level at which the microbubbles burst.

### DETAILED DESCRIPTION OF THE PRESENTLY CONTEMPLATED BEST MODE OF PRACTICING THE INVENTION

From previous research, the applicant herein has found that several characteristics of an unstable plaque may be observed as being present. Although the growth of unstable plaque is accompanied by lipid deposits, proliferation of smooth muscle cells, a shift of smooth muscle cells from a contractile to a secretory type and the presence of leukocytes, and, in the early phase also lymphocytes, and later, monocytic cells such as monocytes and phagocytes, the plaque is nevertheless in a stable condition as long as a fibrous cap remains and is covered by an endothelial layer. The thin endothelial layer assures that no coagulation cascade is occurring inside the vessel. That is, as long as a paving of the endothelial layer is present, the normal blood cells of the coagulation cascade, such as platelets (or thrombocytes), will not adhere to the endothelial structure or to the vessel wall.

When an unstable plaque exists, however, the endothelial layer is fissured or disrupted and sub-endothelial structures are exposed to the blood. As a consequence, several phenomena occur at this site including a fibrin thrombus platelet accumulation, platelet leukocytes interaction, and finally, the build up of a red thrombus. Also, the endothelial paving and the integrity of endothelial function is severely compromised.

According to one aspect of the present invention, tiny bubbles, or microbubbles—being in a range of diameters even smaller than that of normal red blood cells, preferably less than about 6 microns, more preferably under about 3 or 4 microns, and most preferably equal to or less than one micron—are labeled with one or more selected antibodies. This is achieved by attaching specific antibodies or other ligands against the structure of an unstable plaque to the microbubble shell, more specifically to the outer layer of the shell. Preferably, the shell has a first, inner layer of a biocompatible polymer such as PLA, and a second, outer layer of ambiphilic character or protein such as albumin. The antibody is conjugated to the protein layer of the shell by conventional protein chemistry technique. In particular, these are antibodies against monocytes, smooth muscle cells of the contractile type, platelets expressing certain surface markers such as CD62/63 that show they are in an activated state, markers of fibrin deposits and fibrin links between the platelets, antibodies against metalloproteinases 2 and/or 9, and antibodies identifying smooth muscle cells of the secretory type as well as of the contractile type. Antibodies that are expressed by the less-differentiated, productive secretory smooth muscle cells, for example, are typical for arteriosclerotic proliferation.

Further according to the invention, these antibodies-labeled microbubbles are injected into the venous circulation. The extremely small size of the bubbles enables them to undergo capillary passage, which is mandatory for them to proceed through the pulmonary circulation and, following perfusion of the heart, on into the arterial circulation. While circulating through the coronaries, the antibodies conjugated to the bubble's shell will become bound, through preferential attachment to the exposed sub-endothelial structures by reaction with the local antigens. In this way, a strong local deposit of contrast-giving bubbles is built up at the sites of unstable plaque. By allowing at least a predetermined sufficient time interval of circulation, the antibody-labeled bubbles can bind to the antigen-expressing target site.

To increase the ratio of the energy binding the bubble to the target site to the mechanical energy produced by the blood flow acting to dislodge the bound bubble, the diameter of the bubble should be at the lower end of the aforementioned range, i.e., equal to or less than one micron. The shell should be of adequate strength to prevent the gas encapsulated therein from leaking in the normal environment of the patient's circulatory system, and to resist rupture under pressure exerted by the blood when the bubble is injected into the circulatory system, but to rupture when subjected to a predefined level of energy such as that produced by an appropriate ultrasonic wave. The gas contained by encapsulation within the double-layer shell described above should be highly soluble in blood, such as nitrogen or air.

Further, to enhance the signaling ratio of bubbles that are locally bound to the exposed sub-endothelial structures to circulating bubbles constituting background noise, the circulating bubbles are sonicated at a site different from the heart. For example, by sonication of the aorta ascending or descending, the aorta abdominally, a femoral artery, carotid artery, or brachial artery, the vast majority of the bubbles remaining in the respective subsequent circulation are destroyed by exposure to the ultrasound energy. The sonication is preferably performed after expiration of the aforementioned predetermined time interval of circulation in which the antibody-labeled bubbles bind to the antigen-expressing target site. Accordingly, upon echocardiography of the heart only those bubbles that are adherent to the antigen structure are detected, and their presence is indicative of an unstable plaque. Detection is performed, after destroying the still circulating bubbles at a site different from the target site, by subjecting the region of the circulatory system of interest—typically the coronary arteries—to ultrasonic energy of sufficient magnitude to rupture the bound bubbles and thereby produce a reflectance contrast indicative of the site of unstable plaque. In this way, the principles of the present invention enable a simple and efficient non-invasive way to identify patients with unstable plaque, and thus, at increased risk for a MI.

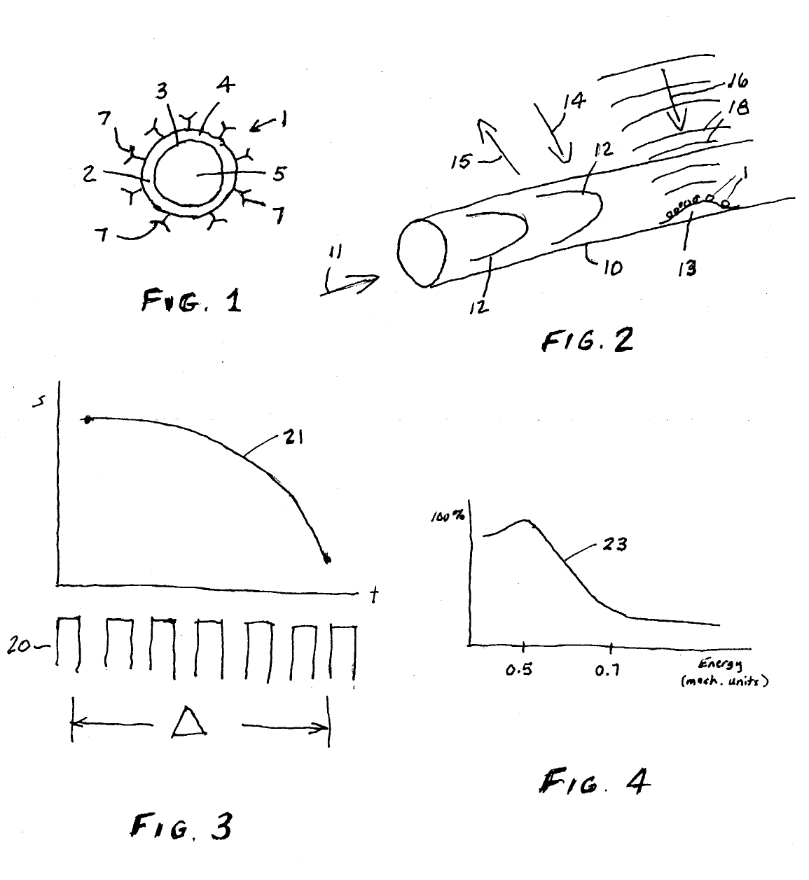

Referring to FIG. 1, each microbubble 1 is formed with a double shell 2, more specifically, a shell having two separate and distinct layers 3, 4. The first, or inner, layer 3 is preferably composed of a polymer such as PLA. The second, or outer, layer 4 is preferably composed of a material having ambiphilic characteristics (both hydrophilic and hydrophobic properties) such as albumin. The spacing shown in the Figure, between the inner and outer layers, is purely for the sake of clarity. In practice, the outer layer lies directly atop the inner layer. Both layers should be capable of withstanding penetration by body fluid, especially the blood (and from leaking of the encapsulated gas to the blood), at least for a time period greater than that which the bubbles will be traversing the circulatory system after injection into the patient's blood stream. The shell should also be capable of withstanding the normal pressure exerted on it when the bubble is carried within the blood flow, and when attaching to a site within a blood vessel or elsewhere in the circulatory system. But the shell should also be adapted to undergo rupture when subjected to a defined mechanical force (or within a defined range of such force) exerted on it when the bubble is subjected to a predetermined level of ultrasonic energy. As previously discussed herein, the overall diameter of the shell 2 (i.e., of the outer layer 4) should be less than 6 μm, and most preferably not greater than 1 μm, so as to provide greater assurance that the bubble has sufficient binding energy to remain bound to a target site in the presence of the mechanical energy exerted by the blood circulation.

A gas 5 is encapsulated within shell 2 for release upon rupture of the shell. The gas must be biocompatible, as is each layer 3, 4 of the shell, and should be highly soluble in body fluid, especially the blood. Also, it should be other than perfluorooxycarbon and is preferably nitrogen or air or other gas that is highly soluble in blood.

The outer layer 4 of the shell 2 is loaded (labeled) with specific antibodies 7 against antigens present at sites designated as likely for unstable plaque. The antibodies, which are schematically depicted in FIG. 1, should be specific against at least one or more of activated platelets (CD62/63), monocytes (CD11b), fibrinogen, metalloproteinases 2 and/or 9, collagen type I, III, IV, elastin, or smooth muscle cells. Two or more different antibodies may be present on the same bubble, such as against platelets and fibrinogen.

The bubbles, or microbubbles, are preferably injected in to the venous circulation for passage through the pulmonary circulation, the heart, and into the circulatory system including the coronary arteries. The tiny bubble size allows their unimpeded passage through the capillaries in the lungs. Referring now to FIG. 2, a portion of the patient's circulatory system is illustrated as a vessel 10, with arrow 11 indicating the direction of blood flow and waves 12 schematically indicating bubbles carried along with the blood circulation, although it will be understood that the bubbles are not separated in groups as the illustrated waves 12 are. At some point in the circulation, one or more sites 13 of pathologic vascular function such as unstable plaque may exist along the vessel wall. The patient will have been designated as a candidate for likelihood of unstable plaque by the attending cardiologist before the procedure to identify sites is commenced, by either clinical characteristics, elevated enzymes, or laboratory blood findings such as elevated inflammatory markers such as CRP, Interleukin 8 or 18, Monocyte Chemoattractant Protein 1 (MCP-1), or increased levels of soluble cell adhesion molecules such as ICAM-1 or VCAM-1 or CD40. Such sites 13 of pathologic vascular function are encountered by the antibody-labeled bubbles 1 present in the blood flow, resulting in a preferential binding thereto of some of these bubbles for interaction of their antibodies with the antigen.

The labeled bubbles may be injected into the venous circulation either by a single bolus injection or by a continuous injection, and are allowed to undergo circulation over a specified sufficient period of time, such as a period in a range from 1 to 10 minutes measured from termination of the injection, so as to permit the desired bubble attachments at the target site(s). One or two additional minutes of circulation may be desirable to allow a firm adhesion of the bubbles at the site where the antigen-antibody interaction takes place. Several bolus injections or more lengthy continuous injection of the bubbles may be needed because the circulatory half time of a bubble is about 90 seconds owing to retention of the bubbles in the reticulo endothelial system (RES) of the liver and spleen. After the specified time period allotted for circulation and firm adhesion of the bubbles, controlled sonication is performed at designated regions of the patient's vena cava or at other all major vessels of the circulatory system that carry large quantities of blood and are not subject to be the suspected target binding site. The purpose of the latter step is to destroy bubbles that have not yet become bound to target sites in the circulatory system. The energy applied through this sonication, by delivery of ultrasound radiation, is at a level sufficient to rupture the unbound bubbles desired to be removed from the equation, to eliminate “noise” that would otherwise mask the specific sites sought to be detected in the identification procedure.

Referring again to FIG. 2, arrows 14 and 15 are intended as a symbolic representation of the applied and reflected ultrasound energy, respectively, into and from the designated high volume blood flow regions in the circulatory system apart from target sites. The desire is then to locate the sites 13 at which large quantities of bubbles 1 are bound. It will be understood by the reader, of course, that the illustration in FIG. 2 is for the sake of convenience and clarity only, and that the target sites normally would not be located in regions of high volume blood flow. The primary areas of interest, especially the coronary arteries, are then subjected to sonication as illustrated by the arrow 16 directed into the site 13. Here again, if this ultrasonic energy level is sufficient to burst the bubbles 1 attached to site 13, the encapsulated gas is released and a detectable site-identification signal is emitted, as indicated by the waves 18 radiating from site 13. The sonication energy can be applied from outside the body, but an inside intraventricular application is also feasible.

FIGS. 3 and 4 aid in illustrating the technique employed, according to the present invention, to apply the ultrasound energy, cause rupture of the bubbles, and detect and identify the target site of unstable plaque with optimum signal-to-noise ratio. As ultrasonic pulses 20 (FIG. 3) are applied to the region(s) of interest (e.g., in the direction of arrow 16 toward site 13 (FIG. 2), bubbles 1 bound to the site 13 being irradiated with the ultrasonic energy begin to burst and the entire complement of attached bubbles rapidly undergo rupture. This results in detection of a rapidly decaying reflectance signal 21 as shown in the graph of reflectance signal strength S versus time t in FIG. 3. The difference ▴ between the detected maximum level of reflectance signal and a predetermined minimum level (>0) reflectance signal represents the optimum bound bubble-emitted (reflectance) signal-to-noise ratio, effectively eliminating background noise from adversely affecting the result, namely identifying the location of the site(s) of unstable plaque.

The defined level of mechanical energy required to burst a bubble is illustrated in the graph of FIG. 4, with bubble population indicated along the y-axis and units of mechanical energy or strength along the x-axis. As the ultrasonic energy is pumped into the region of interest, the bubble population rapidly diminishes according to a curve 23 having the greatest slope between units 0.5 to 0.7 mechanical units.

Although a best mode of practicing the invention has been disclosed by reference to a preferred method, it will be apparent to those skilled in the art from a consideration of the foregoing description that variations and modifications may be made without departing from the spirit and scope of the invention. Accordingly, it is intended that the invention be limited only by the appended claims and the rules and principles of applicable law.

What is claimed is:

1. A method of identifying a patient at increased risk for myocardial infarction, which comprises:
forming microbubbles each consisting of an encapsulated gas characterized by rapid dissolution in blood, and labeling said microbubbles with a prescribed antibody;
injecting said microbubbles into the patient's circulation to allow the microbubbles to preferentially attach themselves to a site of specific antigens in the coronary circulation;
applying ultrasonic energy to the patient's coronary circulation at a level sufficient to burst the microbubbles preferentially attached to said site and allow escape of said gas from the burst microbubbles for absorption by the blood; and
detecting a signal representing reflectance of ultrasonic energy from said site.

2. The method of claim 1, including:
using nitrogen as the gas for the microbubbles.

3. The method of claim 1, including:
forming said microbubbles in sizes less than about 6 microns in diameter.

4. The method of claim 1, including:
encapsulating each of said mirobubbles in a double-layer shell.

5. The method of claim 4, wherin:
said shell comprises an outer ambiphilic layer and an inner polymeric layer.

6. The method of claim 1, including:
exposing a portion of circulation system remote from the coronary circulation region to sonication to destroy microbubbles still circulating after a predetermined period of time, to reduce their number, and thereby, background noise during signal detection before applying ultrasound energy to the site of bubble attachment.

7. The method of claim 6, wherein:
said period of time is in a range from about one minute to about 12 minutes, to enable firm attachment of said preferentially-attaching microbubbles to said site.

8. The method of claim 1, including:
detecting ischemia in the coronary circulation at the same time as unstable plaque detection.

9. A method of identifying within a patient a site of pathologic vascular function in a blood vessel, which comprises:
(a) performing intravascular injection of bubbles consisting of nontoxic gas encapsulated in a rupturable shell, said bubbles being sufficiently small to pass through the patient's capillary bed; said bubbles characterized by
(i) propensity to undergo binding specific to sites of pathologic vascular function in the systemic circulation,
(ii) force by which said bubble is bound to a site of pathologic vascular function exceeds mechanical forces exerted on the bubble by blood flow,
(iii) destruction upon subjection to a defined level of mechanical energy,
(iv) rapid solubility of said gas released upon destruction of the bubble; and
(b) subjecting said bubbles bound to said sites in the blood vessel to a train of ultrasound pulses sufficient to produce said defined level of mechanical energy for rupture of bubbles exposed thereto, and to detect the emission of a signal upon rupture and dissolution of the gas in the blood.

10. The method of claim 9, wherein:
each of said bubbles includes a double layer shell encapsulating said gas.

11. The method of claim 10, wherein:
said shell comprises a polymer inner layer and an albumin outer layer.

12. The method of claim 9, including:
allowing said bubbles to undergo systemic circulation for a period of time sufficient for preferential binding of bubbles to sites of pathologic vascular function in said arteries, and
destroying bubbles still circulating after said period of time by sonication at a site different from the target site to increase the sensitivity for detecting the bound bubbles in the vessel.

13. The method of claim 9, wherein:
the pathologic vascular function is a ruptured plaque.

14. The method of claim 9, wherein:
each of said sites is a site with pathologic endothelial function.

15. The method of claim 9, wherein:
said bubbles are formed to undergo reaction with and binding to activated platelets, with surface markers of either CD62 or CD63.

16. The method of claim 11, wherein:
said albumin outer layer is formed with at least one antibody thereon directed against antigen structures of the unstable plaque.

17. The method of claim 11, wherein:
said albumin outer layer is formed with two or more antibodies directed against the structure of the pathologic vascular function.

18. The method of claim 11, wherein:
said albumin outer layer is formed with antibodies thereon directed against fibrinogen.

19. The method of claim 11, wherein:
said albumin outer layer is formed with antibodies thereon directed against monocytes.

20. The method of claim 11, wherein:
said albumin outer layer is formed with antibodies thereon directed against CD11b.

21. The method of claim 9, including:
enhancing the sensitivity to detect said bubbles bound to said sites by sonication through disruption over a vascular site other than a said pathologic vascular function site and prior to subjecting said bubbles bound to sites to ultrasound pulses.

22. The method of claim 9, wherein:
said bubbles are smaller than 6 μm.

23. The method of claim 9, wherein:
said bubbles are smaller than 3 μm.

24. The method of claim 9, wherein:
said bubbles are smaller than 1 μm.

25. The method of claim 9, including:
applying said ultrasound pulses transthoracically.

26. The method of claim 9, including:
applying said ultrasound pulses from an endovascular site.

27. In a method of ultrasound enhanced detection of unstable plaque in a vessel of a patient's circulation system, performing the steps of:
preparing bubbles in which a gas selected for biocompatibility and solubility in blood is encapsulated in a double layer shell, said shell comprising a first inner layer of a polymer and a second outer layer of albumin;
infusing the surface of said albumin outer layer with antibodies against antigen present in conjunction with structures of unstable plaque;
releasing said bubbles into the circulation for preferential adherence to said antigen structures in said vessel so as to generate signals indicative of presence of said structures, and thereby, of unstable plaque, when said adherent bubbles are ruptured by subjection to predefined levels of ultrasonic energy.

28. In the method of claim 27,
detecting said signals emanating from the rupturing bubbles, by sensing differential decay of said signals over a predetermined time interval.

29. In the method of claim 27, wherein
said infused antibodies are directed against an antigen indicative of monocytes.

30. In the method of claim 29, wherein
said monocyte antibody is CD11b.

31. In the method of claim 27, wherein
said infused antibodies are directed against an antigen indicative of activated platelets.

32. In the method of claim 31, wherein
said activated platelets antibody is selected from CD62 and CD63.

33. In the method of claim 27, wherein
said infused antibodies are directed against an antigen indicative of fibrinogen.

34. In the method of claim 27, wherein
said infused antibodies are directed against an antigen indicative of metalloproteinases.

35. In the method of claim 27,
sonicating circulating bubbles for destruction of some of them during passage through a relatively large vessel.

36. In the method of claim 27,
repetitively scanning with ultrasound in multiple short axis planes to view the majority of the patient's heart for signals indicative of unstable plaque in the coronary arteries.

37. In a method to detect a site of pathologic vascular function in the arterial
circulation system of a patient:
intravascularly applying gas filled echo contrast bubbles imbued with a specific antibody against CD11b on the bubble surface, and
detecting sites at which numbers of said bubbles become bound, by application of ultrasound.

38. In a method to detect a site of pathologic vascular function in the arterial circulation system of a patient:
intravascularly applying gas filled echo contrast bubbles with a specific antibody against CD62 or CD63 on the bubble surface, and
detecting sites at which numbers of said bubbles become bound, by application of ultrasound.

39. In a method to detect a site of pathologic vascular function in the arterial circulation system of a patient:
intravascularly applying gas filled echo contrast bubbles with a specific antibody against more than a single antigen present at said site on the bubble surface, and
detecting said site as a location at which numbers of said bubbles become bound, by application of ultrasound.

40. In the method of claim 39,
evaluating findings of said echo bubble binding in light of systemic blood serum markers of general inflammation CRP, MCP-1, and Interleukins.
